(19) United States
(12) Patent Application Publication
(10) Pub. No.: US 2019/0254579 A1
(43) Pub. Date: Aug. 22, 2019

(54) INTEGRATED DEVICES TO CONTINUOUSLY MEASURE BOUND AND UNBOUND ANALYTE FRACTIONS IN BIOFLUIDS

(71) Applicants: Eccrine Systems, Inc., Cincinnati, OH (US); University of Cincinnati, Cincinnati, OH (US)

(72) Inventors: Jason Heikenfeld, Cincinnati, OH (US); Hector Wong, Cincinnati, OH (US); Kevin Plaxco, Santa Barbara, CA (US); Jacob A. Bertrand, Norwood, OH (US)

(73) Assignees: Eccrine Systems, Inc., Cincinnati, OH (US); University of Cincinnati, Cincinnati, OH (US)

(21) Appl. No.: 16/402,697

(22) Filed: May 3, 2019

Related U.S. Application Data
(63) Continuation-in-part of application No. 15/769,435, filed on Apr. 19, 2018.
(60) Provisional application No. 62/666,921, filed on May 4, 2018.
(60) Provisional application No. 62/775,191, filed on Dec. 4, 2018.
(60) Provisional application No. 62/364,589, filed on Jul. 20, 2016.
(60) Provisional application No. 62/245,638, filed on Oct. 23, 2015.
(60) Provisional application No. 62/269,244, filed on Dec. 18, 2015.
(60) Provisional application No. 62/269,447, filed on Dec. 18, 2015.

(51) Int. Cl. A61B 5/1486; A61B 5/145; A61B 5/1491; G01N 33/53; G01N 27/327

(52) U.S. Cl. CPC A61B 5/14865; A61B 5/14546; A61B 5/14539; G01N 27/3275; A61B 5/14514; G01N 33/5302; G01N 27/3271; A61B 5/1491

(57) ABSTRACT

Embodiments of the disclosed invention provide devices for measuring concentrations of bound and unbound fractions of a target analyte in a biofluid sample. Analytes present in biofluid may be found in a free state, or bound to a binding solute, presenting difficulties for wearable analyte sensors to measure physiologically significant concentrations of the analyte in biofluid. The disclosed devices feature sensors configured to measure both the bound and unbound fractions of the analyte, as well as analyte releasers that cause a portion of the bound fraction of analytes to be released to facilitate measurement. Some embodiments include a collector and or a sample conduit. Other embodiments include a plurality of fluid pathways.

### CROSS-REFERENCE TO RELATED APPLICATIONS

The present application is a Continuation-in-Part of Ser. No. 15/769,435, filed Apr. 19, 2018, and claims priority to U.S. Provisional Application No. 62/666,921, filed May 4, 2018; U.S. Provisional Application No. 62/775,191, filed Dec. 4, 2018; PCT/US16/58357, filed Oct. 23, 2016; U.S. Provisional Application No. 62/364,589, filed Jul. 20, 2016; U.S. Provisional Application No. 62/245,638, filed Oct. 23, 2015; U.S. Provisional No. 62/269,244, filed Dec. 18, 2015; and U.S. Provisional Application No. 62/269,447, filed Dec. 18, 2015, the disclosures of which are hereby incorporated herein by reference in their entirety.

### BACKGROUND OF THE INVENTION

Non-invasive biosensing technologies have enormous potential for several medical, fitness, and personal well-being applications. The sweat ducts can provide a route of access to many of the same biomarkers, chemicals, and other solutes that are carried in blood and can provide significant information enabling one to diagnose ailments, health status, toxins, performance, and other physiological attributes even in advance of any physical sign. Sweat has many of the same analytes and analyte concentrations found in blood and interstitial fluid. Interstitial fluid has even more analytes nearer to blood concentrations than sweat does, especially for larger sized and more hydrophilic analytes (such as proteins).

However, one challenge for both fluids, especially for sweat, is that high-concentration ions such as Na⁺, K⁺, ammonium, Cl⁻, pH, and other chemical solutes in sweat can interfere with sensors specific to analytes such as aptamer sensors for cortisol, or amperometric/ion-selective sensors for urea. The primary issue is that the concentration of these interfering solutes can change over wide ranges. If such solutes were more stable in sweat, the resulting interference could be resolved through calibration or other suitable methods. One possible solution is to measure the solute concentrations in real-time, and to use those measurements to correct the other sensor readings. However, this solution inefficiently uses two sensors to achieve one sensing result, and compounds the individual errors from each sensor. What is needed are simple yet robust methods to chemically buffer a sweat, biofluid, or other fluid sample in a fluid sensing device, ideally without reducing chronologically assured sampling rates.

Continuously measuring analytes in biofluids also brings about additional challenges, for example, there are several challenges with analytes such as hormones, peptides, drugs, and other analytes, that are significantly bound to other biofluid solutes such as proteins. For example, a significant fraction of cortisol is bound in blood, or for example, measurement of drug concentrations is confounded in some cases by the drugs being bound to proteins as taught in “Therapeutic Drug Monitoring in Interstitial Fluid: A Feasibility Study Using a Comprehensive Panel of Drugs” DOI 10.1002/jps.23309. This presents at least two challenges. First this reduces the total measurable concentration of the analyte presented to a biosensor making it more difficult for the biosensor to measure the analyte. Second, understanding how much of the analyte is bound vs. unbound is important in some applications, such as drug development as taught in “Blood Protein Binding of Cyclosporine in Transplant Patients” https://doi.org/10.1002/j.1552-4604.1987.tb02192.x where they state “The results of this study indicate that there are differences in blood protein binding of cyclosporine between transplant patients that may contribute to the differences in the pharmacokinetics and pharmacodynamics of this drug.” This is not an easy challenge to resolve with existing technology such as the most commonly utilized in-dwelling sensors, where a sensor is placed into the dermis of the skin, because the sensor has no capability to liberate bound analytes or to distinctively measure bound vs. unbound fractions of the analyte. This is also a non-obvious problem to solve as distinctly measuring unbound vs. unbound fractions has been primarily limited to laboratory assays, and has not yet been a topic that has been emphasized as an issue to be resolved for continuous peripheral biochemical monitoring. This problem is relevant to general biofluids, and is most relevant to protein rich biofluids such as interstitial fluid and blood.

Many of the challenges stated above can be resolved by if a biofluid can be sampled into a device capable of pre-treating the biofluid before it is sensed by a sensor in order to measure the bound and/or unbound fractions of an analyte.

### SUMMARY OF THE INVENTION

Embodiments of the disclosed invention provide devices for measuring concentrations of bound and unbound fractions of a target analyte in a biofluid sample. Analytes present in biofluid may be found in a free state, or bound to a binding solute, presenting difficulties for wearable analyte sensors to measure physiologically significant concentrations of the analyte in biofluid. The disclosed devices feature sensors configured to measure both the bound and/or unbound fractions of the analyte, as well as analyte releasers that cause a portion of the bound fraction of analytes to be released to facilitate measurement. Some embodiments include a collector and or a sample conduit. Other embodiments include a plurality of fluid pathways.

### BRIEF DESCRIPTION OF THE DRAWINGS

The objects and advantages of the disclosed invention will be further appreciated in light of the following detailed descriptions and drawings in which:

FIG. 1 depicts an embodiment of the disclosed invention configured to provide buffering of fluid samples.

FIG. 2 depicts an embodiment of the disclosed invention configured to provide buffering of fluid samples.

FIG. 3 depicts an embodiment of the disclosed invention configured to provide buffering of fluid samples.

FIG. 4 depicts an embodiment of the disclosed invention configured to provide buffering of fluid samples.

FIG. 5 is a cross-sectional view of an embodiment of the present invention illustrating a device where at least bound fractions of an analyte can be continuously measured by a sensor.

FIG. 6 is a cross-sectional view of an embodiment of the present invention illustrating a device where at least bound fractions of an analyte can be continuously measured by a sensor.

FIG. 7 is a cross-sectional view of an embodiment of the present invention illustrating a device where at least bound fractions of an analyte can be continuously measured by a sensor.

FIG. 8 is a cross-sectional view of an embodiment of the present invention illustrating a device where at least bound fractions of an analyte can be continuously measured by a sensor.

FIG. 9 is a cross-sectional view of an embodiment of the present invention illustrating a device where at least bound fractions of an analyte can be continuously measured by a sensor.

FIG. 10 is a cross-sectional view of an embodiment of the present invention illustrating an implanted device where at least bound fractions of an analyte can be continuously measured by a sensor.

FIG. 11A is a cross-sectional view of at least a portion of a membrane enhanced sensor.

FIG. 11B is another cross-sectional view of at least a portion of a membrane enhanced sensor.

FIG. 12 is cross-sectional view of at least a portion of a membrane enhanced sensor.

FIG. 13 is cross-sectional view of at least a portion of a fully encapsulated sensor.

FIG. 14 is cross-sectional view of at least a portion of the hydrophobic barrier of a membrane enhanced sensor.

### DEFINITIONS

As used herein, “sweat” or “sweat biofluid” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva. A biofluid may be diluted with water or other solvents inside a device because the term biofluid refers to the state of the fluid as it emerges from the body.

As used herein, “fluid” may mean any human biofluid, or other fluid, such as water, including without limitation, groundwater, sea water, freshwater, etc., petroleum products, or other fluids.

As used herein, “continuous monitoring” means the capability of a device to provide at least one sensing and measurement of fluid collected continuously or on multiple occasions, or to provide a plurality of fluid measurements over time.

As used herein, “chronological assurance” is an assurance of the sampling rate for measurement(s) of sweat (or other biofluid or fluid), or solutes in sweat, being the rate at which measurements can be made of new sweat or its new solutes as they originate from the body.

Chronological assurance may also include a determination of the effect of sensor function, or potential contamination with previously generated sweat, previously generated solutes, other fluid, or other measurement contamination sources for the measurement(s).

As used herein, “determined” may encompass more specific meanings including but not limited to: something that is predetermined before use of a device; something that is determined during use of a device; something that could be a combination of determinations made before and during use of a device.

As used herein, “sweat sampling rate” is the effective rate at which new sweat, or sweat solutes, originating from the sweat gland or from skin or tissue, reaches a sensor that measures a property of sweat or its solutes. Sweat sampling rate, in some cases, can be far more complex than just sweat generation rate. Sweat sampling rate directly determines, or is a contributing factor in determining, the chronological assurance. Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval(s)”. Sweat sampling rates or intervals are not necessarily regular, discrete, periodic, discontinuous, or subject to other limitations. Like chronological assurance, sweat sampling rate may also include a determination of the effect of potential contamination with previously generated sweat, previously generated solutes, other fluid, or other measurement contamination sources for the measurement(s). Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

As used herein, “sweat stimulation” is the direct or indirect causing of sweat generation by any external stimulus, the external stimulus being applied for the purpose of stimulating sweat. Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis. A device for iontophoresis may, for example, provide direct current and use large lead electrodes lined with porous material, where the positive pole is dampened with 2% pilocarpine hydrochloride and the negative one with 0.9% NaCl solution. Sweat can also be controlled or created by asking the device wearer to enact or increase activities or conditions that cause them to sweat. These techniques may be referred to as active control of sweat generation rate.

As used herein, “sweat generation rate” is the rate at which sweat is generated by eccrine sweat glands. Sweat generation rate is typically measured by the flow rate from each gland in nL/min/gland. In some cases, the measurement is then multiplied by the number of sweat glands from which sweat is being sampled to calculate the sweat volume sampled per unit time.

As used herein, “fluid sampling rate” is the effective rate at which new fluid, or fluid solutes, originating from the fluid source, reaches a sensor that measures a property of the fluid or its solutes. Fluid sampling rate directly determines, or is a contributing factor in determining, the chronological assurance. Times and rates are inversely proportional (rates having at least partial units of 1/seconds), therefore a short or small time required to refill a fluidic volume can also be said to have a fast or high fluid sampling rate. The inverse of fluid sampling rate (1/s) could also be interpreted as a “fluid sampling interval(s)”. Fluid sampling rates or intervals are not necessarily regular, discrete, periodic, discontinuous, or subject to other limitations. Like chronological assurance, fluid sampling rate may also include a determination of the effect of potential contamination with previously generated fluid, previously generated solutes, other fluid, or other measurement contamination sources for the measurement(s). Fluid sampling rate can also be in whole or in part determined from solute generation, transport, advective transport of fluid, diffusion transport of solutes, or other factors that will impact the rate at which new fluid or fluid solutes reach a sensor and/or are altered by older fluid or solutes or other contamination sources. Sensor response times may also affect sampling rate.

As used herein, “measured” can imply an exact or precise quantitative measurement and can include broader meanings such as, for example, measuring a relative amount of change of something. Measured can also imply a binary measurement, such as ‘yes’ or ‘no’, present/not present type measurements.

As used herein, “fluidic volume” is the fluidic volume in a space that can be defined multiple ways. Fluidic volume may be the volume that exists between a sensor and the point of generation of a fluid or a solute moving into or out of the fluid from the body or from other sources. Fluidic volume can include the volume that can be occupied by fluid between: the sampling site on the skin and a sensor on the skin, where the sensor has no intervening layers, materials, or components between it and the skin; or the sampling site on the skin and a sensor on the skin where there are one or more layers, materials, or components between the sensor and the sampling site on the skin.

As used herein, “solute generation rate” is simply the rate at which solutes move from the body or other sources into a fluid. “Solute sampling rate” includes the rate at which these solutes reach one or more sensors.

As used herein, “microfluidic components” are channels in polymer, textiles, paper, or other components known in the art of microfluidics for guiding movement of a fluid or at least partial containment of a fluid.

As used herein, “state void of fluid” means a fluid sensing device component, such as a space, material or surface, that can be wetted, filled, or partially filled by fluid, when the component is entirely or substantially (e.g., >50%) dry or void of fluid.

As used herein, “advective transport” is a transport mechanism of a substance, or conserved property by a fluid, that is due to the fluid's bulk motion.

As used herein, “diffusion” is the net movement of a substance from a region of high concentration to a region of low concentration. This is also referred to as the movement of a substance down a concentration gradient.

As used herein, a “sample concentrator” is any portion of a device, material, subsystem, or other component that can be utilized to increase the molarity of at least one fluid analyte, at least in part by removing a portion of the water that was originally with the at least one analyte when it exited the body.

As used herein, the term “analyte-specific sensor” is a sensor that performs specific chemical recognition of an analyte's presence or concentration (e.g., ion-selective electrodes, enzymatic sensors, electrochemical aptamer-based sensors, etc.). For example, sensors that sense impedance or conductance of a fluid, such as sweat, are excluded from the definition of analyte-specific sensor because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. Such sensors can be in the forms disclosed in U.S. Pat. Nos. 7,803,542 and 8,003,374 (the “Multi-capture Aptamer Sensor” (MCAS)), or in U.S. Provisional Application No. 62/523,835 (the “Docked Aptamer Sensor” (DAS)).

As used herein, “sample volume” is the fluidic volume in a space that can be defined multiple ways. Sample volume may be the volume that exists between a sensor and the point of generation of a fluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

As used herein, a “volume-reduced pathway” or “reduced-volume pathway” is at least a portion of a sample volume that has been reduced by addition of a material, device, layer, or other component, which therefore increases the sampling interval for a given sample generation rate. A volume-reduced pathway can be created by at least one volume reducing component.

As used herein, “buffering component” is any component that regulates concentration of at least one chemical in the collected sample preferably within at least 20% of a target concentration of at least one chemical, and less preferably within at least 100% or at least 300% of a target concentration.

As used herein, “reversible sensor” means a sensor configured to measure both increasing and decreasing concentrations of a target analyte without any additional change in stimulus or environment for the sensor, other than the change in the analyte concentration.

As used herein, “binding-solute” is a solute in a biofluid or fluid that binds at least one analyte.

As used herein, “analyte releaser” is any component capable of releasing an analyte that is bound to binding-solute in a biofluid or solution.

As used herein, “collector” or “biofluid collector” is any material, cavity, or other device feature that is able to sample, transport, or position a sample of a biofluid into or adjacent to a device such that an analyte releaser can release a target analyte that is bound to other binding-solutes in the solution within proximity of an analyte specific sensor such that the analyte specific sensor is able to measure the increased unbound fraction of the analyte caused by the analyte releaser. A biofluid collector does not necessarily require continuous flow of sample.

As used herein, “sample conduit” includes any material, geometry, channel, feature, or combinations thereof that enables advective flow of a biofluid and/or which supports transport of an analyte through the fluid.

As used herein, “binding-solute filter” is a selectively permeable membrane or other material or component that blocks, traps, or prevents further passage of at least one binding-solute that binds a target analyte. Using a chemical such as an antibody to trap the binding-solute or adding a chemical causing binding solute to precipitate out of solution also therefore acts as a binding-solute filter.

As used herein, “analyte-permeable filter” is a selectively permeable membrane that allows a specific analyte in a solution to pass through, but which blocks transport of at least some other solutes. An analyte permeable filter can also be a “binding-solute filter”.

As used herein, “hydrophobic barrier” is an analyte-permeable filter through which the analyte will diffuse, but which blocks charged or hydrophilic solutes that interfere with or degrade a sensor (hereinafter “interfering solutes”). For example, hydrocarbons or vegetable oils can allow a hydrophobic analyte such as ethanol, cortisol, or acetaminophen, to diffuse through them, but block interfering solutes, such as ions, potential of hydrogen (pH)-altering solutes (acids, bases, H⁺, OH⁻), and other charged or hydrophilic species. Hydrophobic barriers may be liquid, semi-solid or solid, e.g., oils, layers of hydrocarbons, silicone greases, or polymers. A hydrophobic barrier may also be defined as a material with a permeability coefficient (cm/s) for at least one interfering solute that is at least one of the following: greater than (>) 3×, >10×, >100× or >1000× lower than the permeability coefficient for at least one target analyte, e.g., ethanol, cortisol, acetaminophen, or cyclosporin A. A hydrophobic barrier is not a simple size-selective membrane, such as a hydrogel.

As used herein, “pump” is any component capable of providing advective flow of a biofluid, a fluid, or for transporting an analyte or binding solute. A pump could be a syringe, a wicking hydrogel, an electrode for moving charged analytes by iontophoresis, or other suitable structures or methods.

As used herein, “sensor solution” refers to materials through which an analyte will diffuse and in which a sensor is bathed, contained, or which partially forms the sensor. For example, an electrochemical aptamer sensor could be bathed in a sensor solution containing a pH buffer, a salt, and a preservative. As another example, instead of being bathed in a solution, a sensor may be combined with a molecular-imprinted polymer that contains within its porous network a sensor solution with a pH buffer and/or salt solutes.

As used herein, “sample solution” refers to any liquid or fluid which contains at least one analyte that is to be measured in presence, change, concentration, or other measurement, by a sensor specific to that analyte. A sample or sample solution may be a biofluid, but could also be water from the environment, manufacturing fluid for food, or other types of sample solutions that would benefit from the disclosed invention.

### DETAILED DESCRIPTION OF THE INVENTION

Embodiments of the disclosed invention apply at least to any type of fluid sensing device that measures at least one analyte in sweat, interstitial fluid, other biofluid, or other fluid. Further, embodiments of the disclosed invention apply to sensing devices which measure samples at chronologically assured sampling rates or intervals. Further, embodiments of the disclosed invention apply to sensing devices which can take on forms including patches, bands, straps, portions of clothing, wearables, or any suitable mechanism that reliably brings sampling and sensing technology into intimate proximity with a fluid sample as it is transported to the skin surface. While some embodiments of the disclosed invention utilize adhesives to hold the device near the skin, devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Certain embodiments of the disclosed invention show sub-components of what would be sensing devices with more sub-components needed for use of the device in various applications, which are obvious (such as a battery), and for purposes of brevity and of greater focus on inventive aspects, such components are not explicitly shown in the diagrams or described in the embodiments of the disclosed invention.

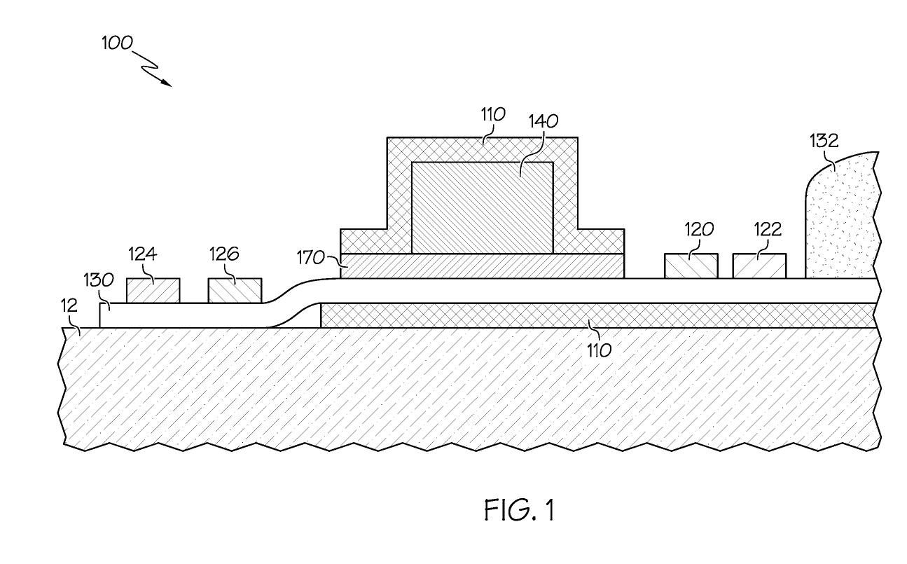

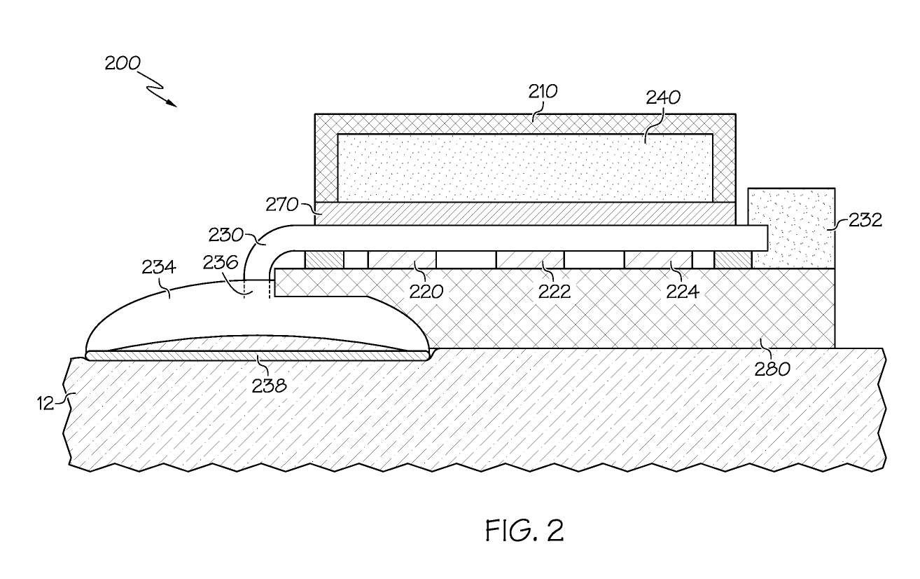

With reference to FIG. 1, in a disclosed embodiment, at least a portion of a fluid sensing device 100 is shown and positioned on the skin 12. The device 100 includes one or more primary sensors 120, 122, and may also include one or more reference sensors 124, 126. The device further includes a polymer substrate 110 and polymer casing 110 made of PET or other suitable material. A fluid collector 130 carries sweat from skin 12 to sensors 120, 122, 124, 126, and onto a fluid sample pump 132 by any suitable mechanism for transport, including osmosis, positive pressure from sweat ducts, or wicking pressures. In some embodiments, the collector 130 and sample pump 132 could be paper or textile wicks. In some embodiments, the collector 130 may include separate collection and transport components (see, e.g., FIG. 2). For example, the fluid collector 130 may include a microchannel that is in fluidic communication with a sample collection component, sensors and sample pump (not shown). The sample pump 232 may be comprised of a desiccant, a sponge, a hydrogel, or other suitable material capable of drawing a fluid sample through the fluid collector, and/or absorbing fluid after it has interacted with the sensors. The device further includes a chemical buffering fluid, gel, solid, or other material 140 and a membrane 170 which, along with casing 110, forms a buffering component.

In some embodiments, the fluid collector 130 itself may perform sample buffering, and in such configurations, a separate buffering component may not be required. The buffering fluid collector may be impregnated with buffering chemicals, or may be chemically modified to provide buffering to the fluid sample as it passes through the fluid collector. For example, the fluid collector could include an ion exchange resin, which would be configured to reduce the concentration of ions that could interfere with the particular measurements needed for a fluid sensing device application. In other embodiments, the buffering fluid collector may be used in combination with a separate buffering component.

In an example embodiment, the primary sensors 120 and 122 are electrochemical aptamer-based (“EAB”) sensors for hormones, which responses will vary with, for example, changes in fluid concentrations of Na⁺ and Cl⁻ (salinity), and H⁺ (pH). For a more complete discussion of EAB sensor variance with salinity and pH, see PCT/US17/23399, which is hereby incorporated by reference herein in its entirety. The buffering component contains, for example, a buffering fluid having 40 mM Na⁺, a pH of 7, and has a fluid volume that is at least one hundred times, or at least one thousand times, or at least ten thousand times greater than the fluid volume of fluid collector 130, e.g., a buffering component volume of 10 μL. In various embodiments, the buffering component may contain a reagent, NaCl, KCl, pH, urea, ammonia, lactate, a reference analyte, or a target analyte. Membrane 170 could be, for example, a PVC polymer membrane embedded with ionophores for Na⁺, Cl⁻, and pH, or just one of these, such that the membrane is relatively impermeable but is more permeable to the chemicals to be buffered. In some embodiments, membrane 170 may be a dialysis membrane. Due to diffusion across the membrane, the fluid sample salinity is stabilized at around 30 mM and pH is stabilized near 7, as the fluid sample reaches the primary sensors 120, 122. In other embodiments, the device may include additional buffering components, each with one or more chemicals and membranes.

With further reference to FIG. 1, several enhancements are possible for device 100. The reference sensors 124, 126, could be analyte-specific sensors for the fluid solutes to be buffered. For example, if the buffering component were imperfect at regulating concentrations in some circumstances (e.g., at very high sweat rates) then the reference sensors 124, 126 could be used to correct for variations in the primary sensors 120, 122 caused by the buffering of the solutes. A biofluid such as sweat also contains many other chemical constituents. If such constituents are not in the buffering component, then water (if that is the fluid in the buffering component) would favor transport by osmosis out of the buffering component and into the sensing area. Therefore, in the disclosed invention, the buffering component may contain an artificial fluid, such as an artificial sweat formulation, that contains concentrations of a plurality of analytes to mitigate such osmosis.

In another embodiment, the disclosed invention may combine the buffering component with a sample concentration component. For further description of sample concentration devices and methods, see PCT/US16/58356, which is hereby incorporated by reference herein in its entirety. In some embodiments, a sample concentration component and a buffering component could be the same component. For example, with reference to FIG. 2, where like numerals refer to like features of previous figures, a device 200 of the disclosed invention is built upon a substrate 280. The device includes a combined buffering and sample concentration component 210, that may include a forward osmosis membrane 270 for concentrating a sample with respect to a target analyte (e.g., cortisol), and a buffering concentrator (“BC”) solution or material 240, which may be, e.g., a disaccharide. The BC solution 240 also contains a 20 mM concentration of NaCl and is at a pH of 7. The membrane 270 allows NaCl and H⁺ to flow freely through, therefore regulating the NaCl and pH in the fluid sample as it flows from skin 12, through fluid microchannel 230 to the sensors 220, 222, 224 and into the fluid sample pump 232. In various embodiments, the device may regulate the concentration of a solute in the fluid sample to within at least 20%, to within at least 100%, or to within at least 300% of a target concentration of the solute.

With further reference to FIG. 2, an embodiment of the device includes a sweat collector 234 having a determined area of contact with a wearer's skin 12. The sweat collector 234 presents a concave shape toward the skin, and has sufficient clearance from skin so that when the device is worn on the body sweat can flow into the device at a natural sweat rate. When applied to a wearer's skin, some of the skin will bulge into the collection area, which aids in providing a seal with skin, but also potentially occludes sweating if the collector is allowed to apply pressure to the sweat ducts. See Johnson, C., et al., “The use of partial sweat duct occlusion in the elucidation of sweat duct function in health and disease,” J. Soc. Cosmet. Chem. 24 15-29 (1973). Some embodiments may include internal ridges (not shown) to maintain space for sweat to flow to the inlet 236. The inlet 236 is in fluidic communication with the microchannel 230, and hence the sensors 220, 222, 224, and sample pump 232. The sweat collector also includes a flexible sealing component 238, which is, for example, a latex or rubber o-ring, a screen-printed silicone gasket, flexible injection-molded ridge, or other suitable material. The sealing component 238 is configured to prevent sweat entering the collection area from the surrounding skin, and to reduce contamination from the skin surface when the device shifts position during normal wear.

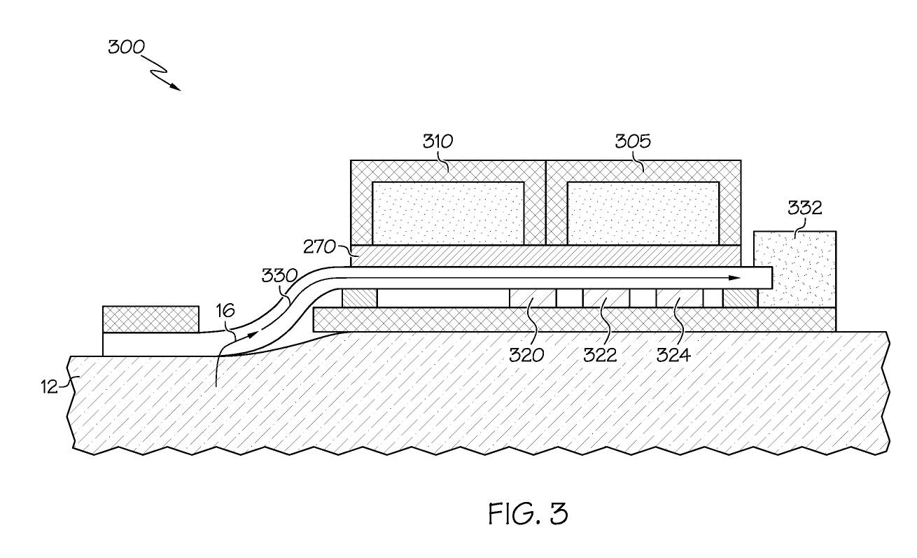

With reference to FIG. 3, where like numerals refer to like features of previous figures, in a preferred embodiment, the buffering component 305 is located after the sample concentration component 310 in relation to the flow direction of the fluid sample 16, so that the fluid is concentrated first, and buffered second. This is because many sample concentration component embodiments could increase salinity, change pH, or concentrate larger acids, bases, or other chemicals that could distort sensor signals. In other embodiments, a buffering component could be configured before a sample concentration component in relation to the flow of the fluid sample, so that the fluid sample is buffered first and concentrated second. In this embodiment, the buffering component could establish a known concentration, such as salinity, which would allow the sample concentration component to have a more predictable degree of sample concentration. For example, a buffering component could regulate the concentration of NaCl to 20 mM and the sample concentration component could have 200 mM draw solution to therefore create a more predictable 10× concentration of the fluid sample before it reaches the analyte-specific sensors.

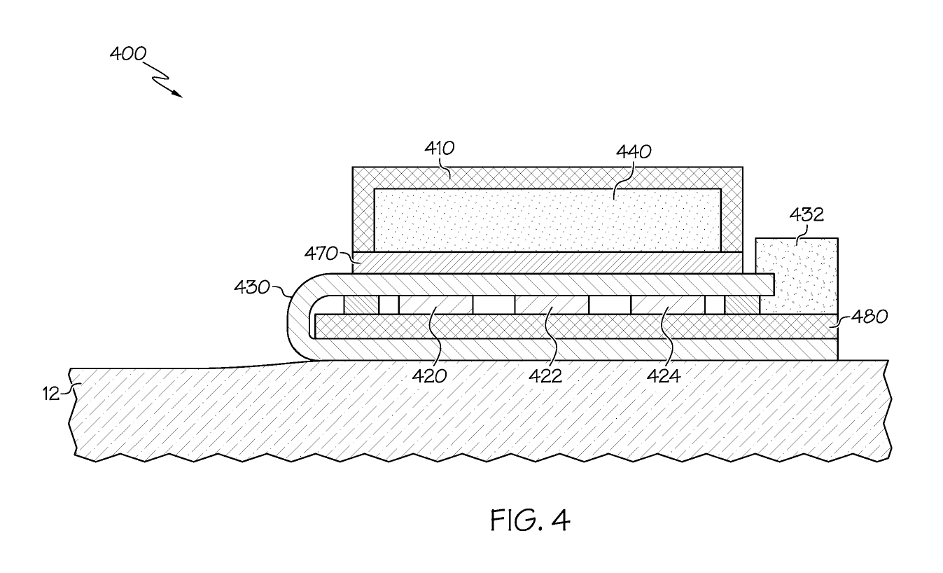

With reference to FIG. 4, where like numerals refer to like features of previous figures, a device 400 of the disclosed invention that is configured for use in a fluid sample is depicted. In this embodiment, the fluid collector 430 is constructed of, e.g., sponge material, and is placed in fluidic communication with a fluid source. For example, the fluid collector 430 could be placed in a container of drinking water (not shown). The device includes a buffering component 410, that may include a forward osmosis membrane 470 for concentrating a sample with respect to a target analyte (e.g., Cryptosporidium or one of that organism's products or toxins), and a buffering solution or material 440, which may contain a 20 mM concentration of NaCl and is at a pH of 7. The membrane 470 allows NaCl and pH to flow freely through, therefore regulating the NaCl and pH in the fluid sample as it is absorbed from the drinking water container, flows through fluid collector 430 to the sensors 420, 422, 424, and then into the fluid sample pump 432.

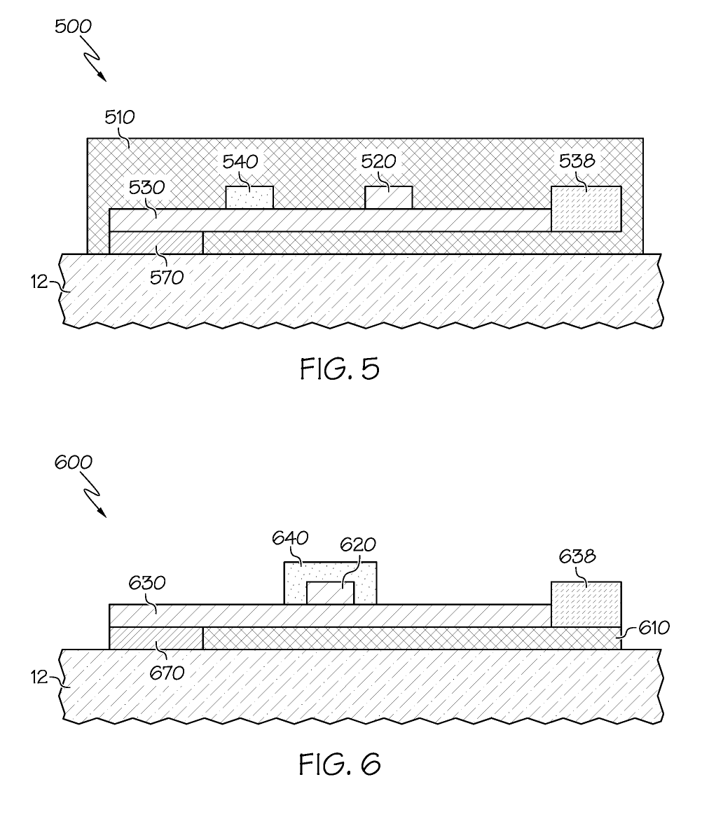

With reference to FIG. 5, a device 500 is placed on or near skin, the lining of the oral cavity, or other surface providing access to a biofluid, e.g., interstitial fluid, sweat, saliva, or blood, such as an artery wall, herein referenced as surface 12. The device 500 is at least partially enclosed or supported by a substrate 510 that is made from silicone, ABS, PET, metal, or other suitable material. The device includes a biofluid sample collector 570 which in some embodiments is a hollow microneedle array for collecting interstitial fluid or blood from an artery, an iontophoresis electrode and hydrogel with a carbachol sweat stimulant for collecting sweat, or other suitable means to provide biofluid access. A sample conduit 530 is in fluidic communication with the collector 570, receives a biofluid sample from the collector 570, and together form a collector conduit 530/570. The sample conduit could be an open-microfluidic film, a closed channel, a wicking material, e.g., paper or rayon, or another suitable material. At least one analyte releaser 540 is coupled to the collector conduit 530/570, and is able to release at least one analyte bound to a binding solute in the biofluid. The analyte releaser 540, depending on the analyte and the binding solute for the analyte, provides a thermal, chemical, mechanical, optical, electrical, or other stimulus to release one or more of greater than (>) 30%, >60%, >90%, or >95% of the analyte that would otherwise be bound to the binding solute in the biofluid. For example, the analyte releaser 540 could be an electric heater; a thermoelectric cooler; a dissolving material, e.g., a tablet of polyvinyl alcohol with TRIS or acetic acid that regulates pH; a reservoir with a selectively permeable membrane containing an acid, base, or other pH buffer to change biofluid pH, e.g., acetic acid, a salt, e.g., NaCl, or a solvent, like water or ethanol; a source of energy, such as ultrasonication transducer, a short-wavelength LED source, or an electrode that alters biofluid pH; or other suitable stimulus to cause the target analyte to dissociate from the biofluid sample. The analyte releaser 540 could be a syringe pump that slowly injects an acidic solution into sample conduit 530, but preferably is less bulky, especially to facilitate wearable applications, and comprises a total volume of one of the following: less than (<) 10 mL, <3 mL, <1 mL, <0.3 mL, or <0.1 mL. For example, the analyte releaser could be a timed-release dissolvable tablet like those used for oral drug delivery. Furthermore, the sustained operation of the analyte releaser is at least one of >1 hour, >12 hours, >24 hours, or >3 days. At least one analyte-specific sensor 520 is coupled to the collector conduit 530/570, and is configured to sense the unbound (released) analyte concentration present in the biofluid. The sensor 520 is, e.g., an electrochemical sensor, or more specifically, an electrochemical aptamer-based (EAB) sensor. In some embodiments, the sensor type may limit the suitable configurations of analyte releaser 540. For example, if the analyte releaser altered biofluid sample pH, an EAB sensor might not function properly in the new pH, or may have to compensate for the altered pH. Solutions to such issues will be taught in later embodiments. A pump 538 at least partially draws the biofluid sample through the device 500, and is, e.g., a wicking hydrogel, such as sodium polyacrylate, a stack of paper, a mechanical pump, a vacuum source, or other suitable pumping means.

With further reference to FIG. 5, an analyte releaser 540 that functions by adding a solvent to the biofluid sample can function in multiple ways. For example, a solvent such as ethanol can alter the folding behavior of a binding solute, e.g., a protein, and thereby causes the analyte to release. Similarly, ethanol may compete with a target analyte for binding sites on the binding solute, causing the analyte to release. A solvent can also cause analyte release though simple dilution. For example, consider a drug analyte that is at 100 μM concentration in the biofluid, and that has a binding affinity with the binding solute centered at 10 μM concentration. Analyte releaser 540 may be a tube that introduces water to the biofluid sample, diluting the analyte concentration by 100× to 100 nM, causing the analyte to release from the binding solute because its concentration is now below the binding affinity. Such techniques would require device sensors 520 to have a limit of detection enabling analyte measurement at the lower post-dilution concentrations.

With reference to FIG. 6, where like numerals refer to like features found in FIG. 5, a device 600 is configured so that an analyte releaser 640 is co-located with an analyte-specific sensor 620. Such embodiments allow the analyte releaser to be more effective, take up less space, consume less power, output less energy, achieve analyte release for a longer duration without unwanted effects on a device wearer, or other advantages. For example, if the analyte releaser applied heat to release the target analyte from its binding solute, to achieve a sustained effect, the heat would need to be sufficient to denature the binding solute. If the analyte releaser were located a distance away from the sensor, this amount of heat may require a prohibitive amount of energy, or cause skin discomfort. Therefore, the analyte releaser 640 and sensor 620 are co-located. In another example, the sensor 620 is encapsulated in a hydrogel that comprises the releasing component 640, wherein the hydrogel is configured to locally change the effective pH or the electrostatic interactions experienced by the binding solute, either of which causes the analyte to release.

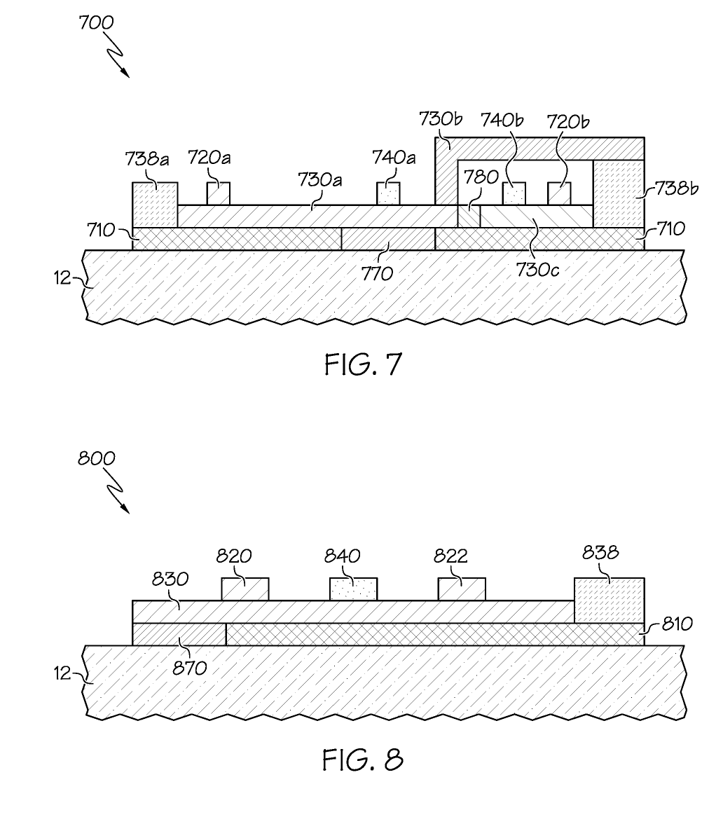

With reference to FIG. 7, where like numerals refer to like features found in FIGS. 5 and 6, a device 700 includes a plurality of distinct fluid pathways 730a, 730c, one or more of which has an analyte releaser 740a, 740b. As a biofluid sample is collected by the collector 770 and moves through the device, it is transported along the first fluid pathway 730a toward the pump 738a. For example, the first pathway 730a, analyte releaser 740a, and first sensor 720a measure the total concentration (bound and unbound concentrations) of an analyte, e.g., cortisol, in a biofluid, e.g., interstitial fluid. Between the first pathway 730a and the second pathway 730c, is at least one binding solute filter 780 (such as a 5 kDa nano-filtration membrane) that blocks the analyte binding solute (e.g., transcortin) and allows transport of the unbound cortisol so that the second sensor 720b only measures the unbound fraction of the analyte. Therefore, the disclosed invention may also include at least one filter that is impermeable to bound analyte, but permeable to unbound analyte. Analyte releaser 740b is optional, and may be included if, for example, the sensors 720a, 720b are pH sensitive and the analyte releasers 740a, 740b alter pH oppositely. A third pathway 730b is provided to cause biofluid, binding solute, and other solutes to bypass the second sensor 720b and to prevent thereby the accumulation of fluid or solutes in the pathways, which may cause degraded transport through the device 700. The disclosed invention therefore may include a plurality of fluid transport paths or sample conduits, and at least one first sensor that measures total concentration of an analyte and at least one second sensor that measures unbound concentration of an analyte. Further, by comparing the measured total and unbound fractions of analyte, the device is capable of determining bound analyte concentration as well. Therefore, the present invention is capable of determining continuously the, bound, unbound, and total concentrations of an analyte.

With reference to FIG. 8, where like numerals refer to like features found in FIGS. 5-7, a device 800 includes a first sensor 820 in series with an analyte releaser 840 in series with a second sensor 822 wherein the first sensor 820 measures unbound concentration of an analyte and the second sensor 822 measures both unbound and bound (total) concentration of an analyte. A capability similar to the device taught in FIG. 7 is achieved. In some embodiments, sensors 820 and 822 may also measure different analytes. For example the first sensor 820 is for vasopressin and the second sensor 822 is for cortisol, which analytes together are markers for dehydration and/or stress. Therefore, the disclosed device may include a plurality of sensors and the ability to sense a plurality of distinct analytes. The first sensor 820 may also be placed downstream of the analyte releaser (not shown) and therefore the disclosed invention includes a plurality of sensors configured to measure a plurality of different analytes in their unbound fractions.

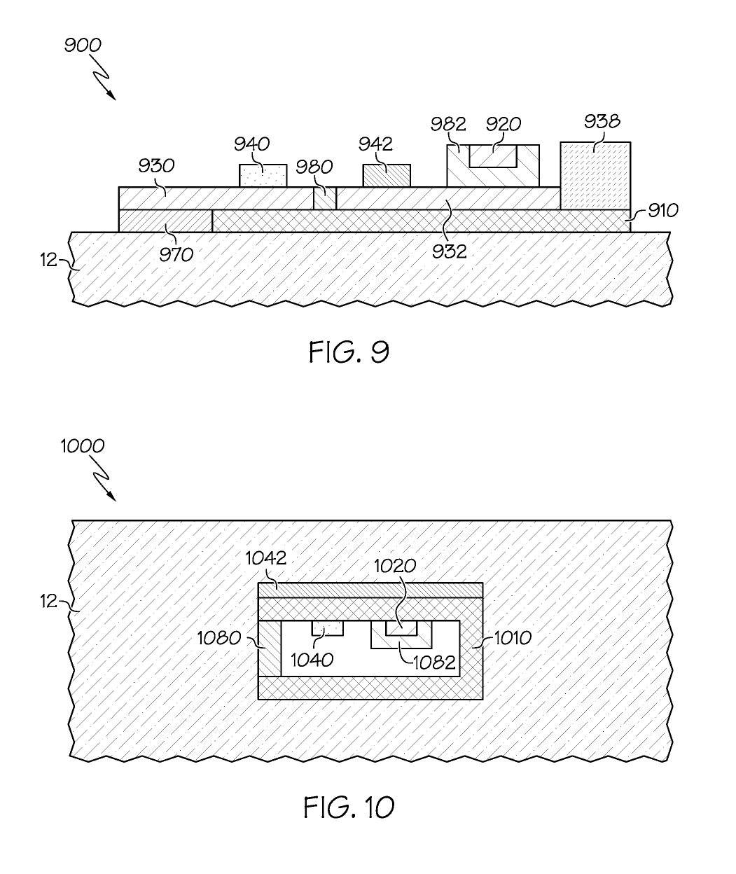

With reference to FIG. 9, where like numerals refer to like features found in FIGS. 5-8, a device 900 includes at least one component to protect the sensor 920 from the effects of the analyte releaser 940. For example, the analyte releaser 940 could alter the biofluid sample pH to a value that harms the sensor 920, or distorts sensor function. Two illustrative examples are provided. In a first example, an optional filter 980 blocks the binding solute but allows the (now unbound) target analyte to pass through. Then, a reverter 942 reverses or mitigates the effect on the biofluid created by the analyte releaser (such as a change in pH) or regulates the biofluid toward conditions preferred by the sensor (e.g., a pH of 7 for sweat biofluid sensing) to facilitate proper sensor operation in the biofluid. Downstream of the filter 980, the target analyte is no longer in the presence of the binding solute, and so will remain unbound despite the reverter's effect on the biofluid. Therefore, the disclosed invention includes at least one component (in this example, the filter 980) that keeps the analyte unbound even if the effect of the analyte releaser is reversed or mitigated, or if the biofluid is otherwise altered by the reverter.

In a second example, the sensor 920 is protected by at least one membrane 982 that is configured to prevent the analyte releaser's effect on the biofluid from reaching sensor 920. For example, if the analyte were cortisol, the membrane 982 could be a hydrophobic barrier that allows cortisol to pass through to the sensor 920, but blocks changes in pH or salinity caused by the analyte releaser 940. These examples may also allow the measurement of analytes that become ionized at certain pH values. For example, tobramycin, which ionizes at blood pH values, could become unbound through action by an analyte releaser 940, and then a reverter 942 alters pH so that tobramycin un-ionizes allowing it to pass through a hydrophobic barrier 982 that protects a sensor. This and similar embodiments do not require a filter 980 in the sample conduit between the analyte releaser and the sensor.

With reference to FIG. 10, where like numerals refer to like features found in FIGS. 5-9, a device 1000 can be indwelling, ranging from partially indwelling (e.g., a needle) to fully indwelling (e.g., a capsule implanted in the body or skin 12). The device includes a large filtration membrane 1080 to keep out cellular and other large debris. The device includes an analyte releaser 1040. In an example embodiment, the analyte releaser 1040 is an electrode with a counter-electrode 1042 configured so that the application of voltage and current locally alters the pH of the biofluid adjacent to the sensor 1020 and inside the device 1000, causing the analyte to release from the binding solute. Sensor 1020 may also be protected by at least one membrane or reverter 1082 so that the effect on the biofluid produced by the analyte releaser (such as a pH change) does not reach the sensor 1020. For example, if the target analyte is cortisol, the membrane 1082 could be a hydrophobic track etch membrane infused with castor oil. The membrane as configured would allow cortisol to pass through to the sensor 1020, but would prevent changes in pH or salinity caused by analyte releaser 1040 from affecting the sensor. In use, such an embodiment may be used to collect an amount of binding-solute, free analyte, and bound analyte. When the analyte releaser is activated, e.g., by pulsed, periodic, or a one-time activation, it causes pH to abruptly change, allowing the sensor to measure the total released analyte concentration. Achieving continuous sensing by this method would be difficult, but repeated sensing is enabled since the larger binding solutes would diffuse into the device slowly, but the smaller released analyte would diffuse out of the device relatively quickly. Such behavior makes it challenging to retain for a long time the released analyte in the device at its total concentration, which is higher than the unbound analyte concentration outside of the device, i.e., the analyte would continually tend to diffuse out of the device. As illustrated by this embodiment, the disclosed invention can be used for multiple applications, and does not always require a pump. However, implanted embodiments could use a pump, for example, to move interstitial fluid into the device, and may leverage one or more of the techniques taught previously for FIGS. 5-9, thereby also allowing continuous sensing of at least the bound fraction of the target analyte.

Devices of the disclosed invention may also provide single-use measurements of bound, unbound, and/or total fractions of a target analyte, for example, as point-of-care testing devices that measure a blood sample obtained by finger-prick.

It may be possible to circumvent the spirit of the disclosed invention by releasing a bound solute, binding the solute to an antibody to characterize it as “bound”, and then detecting the solute-antibody complex using a competitive sandwich assay technique. However, as long as a device or method continuously or repeatedly samples a biofluid, continuously or repeatedly causes a target analyte to release from a binding solute, and then continuously or repeatedly senses the analyte (regardless of the final bound or unbound state of the analyte), then the device or method includes an embodiment of the disclosed invention.

The following examples are provided to help illustrate the present invention, and are not comprehensive or limiting in any manner.

Laboratory examples (not integrated devices) for how to release cortisol from transcortin are taught in “Studies of human transcortin at different pHs: circular dichroism, polymerization and binding affinity,” https://doi.org/10.1016/0014-5793(76)80309-2; “The chemistry of human transcortin: The effects of pH, urea, salt, and temperature on the binding of cortisol and progesterone,” https://doi.org/10.1016/0003-9861(77)90524-0; and “How Changes in Affinity of Corticosteroid-binding Globulin Modulate Free Cortisol Concentration,” https://doi.org/10.1210/jc.2012-4280. The disclosed devices and methods may also be used to measure other analytes, such as protein-bound small molecule drugs. Example electrochemical sensors for cortisol include those taught in “Recent advances in cortisol sensing technologies for point-of-care application,” http://dx.doi.org/10.1016/j.bios.2013.09.060. Small molecule drug detection is also taught in “Real-Time, Aptamer-Based Tracking of Circulating Therapeutic Agents in Living Animals,” DOI: 10.1126/scitranslmed.3007095.

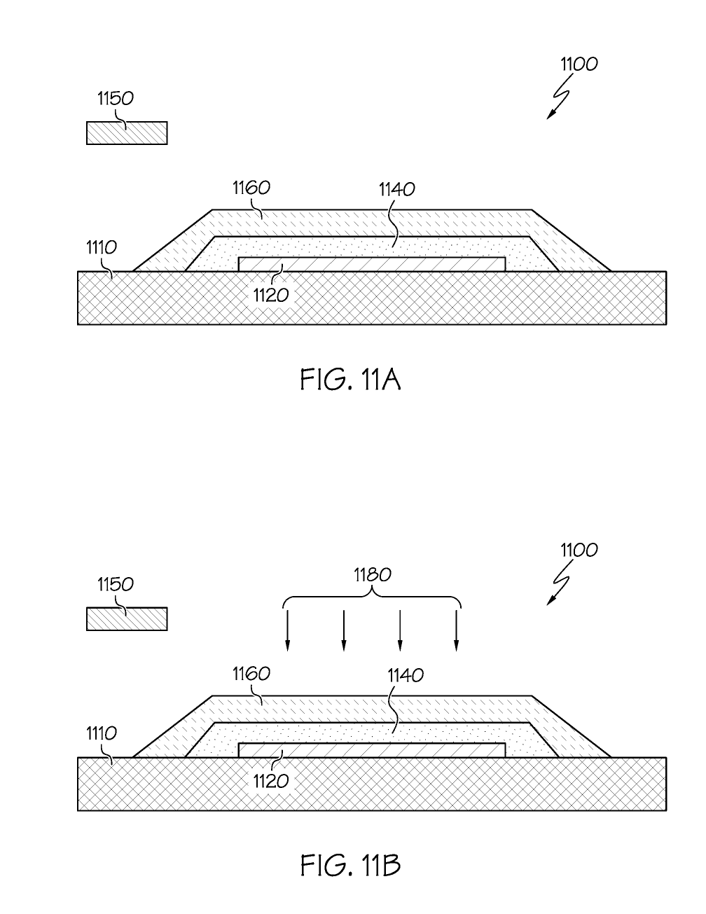

With reference to FIG. 11A, a device 1100 includes a substrate 1110, an analyte specific sensor 1120, a sensor solution 1140, a hydrophobic barrier 1160, and an electrode 1150. The device may be placed into or adjacent to a sample solution 1180 as shown in FIG. 11B. The substrate is any material suitable for supporting the sensor and is typically a solid and inert material. Exemplary substrates may be comprised of glass or PET. The electrode may be for example, a counter electrode of silver, silver chloride, gold, carbon, poly(3,4-ethylenedioxythiophene) (PEDOT), or other materials suitable to function as an electrode. At least one analyte specific sensor is capable of detecting a molecule of interest and may be optical, mechanical, electronic or other suitable means of sensing as indicated above.

Sensor solution 1140 is configured to support diffusion of the target analyte into fluidic communication with the sensor, and support reliable operation of the sensor 1120. For example, the solvent in the sensor solution could be water, a glycol, an alcohol, an ionic liquid, an oil, or other suitable liquid or fluid. The solvent may contain solutes. For example, an aqueous solvent could contain sucrose, a redox moiety, e.g., methylene blue, a salt, e.g., potassium chloride, a buffer, e.g., citrate or 10 mM tris(hydroxymethyl)aminomethane (Tris) and HCl to bring pH to 8.0, a preservative, or any combination thereof, or one or more different solutes or solute types. For example, the pH of sensor solution could be controlled in the solvent such that the pH is always greater than 7, or near 7. Alternatively, the salt concentration can be controlled so that a chloride ion content of the sensor solution is always greater than 10 mM.

Hydrophobic barrier 1160 is able to pass at least one target analyte to at least one sensor 1120 specific to the target analyte, and is able to block at least one interfering solute in a sample solution 1150. The hydrophobic barrier is a material that has a permeability coefficient (cm/s) for at least one interfering solute, where the permeability coefficient for the at least one interfering solute is at least one of >10×, >100× or >1000× lower than the permeability coefficient for at least one target analyte.

For example, hydrophobic barrier could be layers of hydrocarbons or vegetable oils that allow a hydrophobic analyte to diffuse through them, but block ions, such as K⁺, Na⁺, Cl⁻, OH⁻, H⁺ (i.e., the barrier can block pH-altering solutes). Hydrophobic barriers may also be semi-solid or solid, such as silicone greases or polymers. Each analyte may have a different hydrophobic barrier that is ideal for that analyte or device application, which may be characterized in terms of properties such as surface tension, solubility limits, log₁₀(Partition Coefficient) (log P), thickness, porosity, solutes, surfactants, a plurality of miscible or immiscible hydrophobic materials, lag times, etc. In general, a well-designed or ideal hydrophobic barrier for a particular target analyte will have properties that 1) facilitate analyte partitioning from the sample solution 1180 into the barrier; e.g., by reducing the required time and/or energy; 2) facilitate analyte diffusion through the hydrophobic barrier, e.g., by reducing the required time and/or viscosity; and 3) facilitate analyte partitioning from the hydrophobic barrier into the sensor solution 1140 and to the sensor, e.g., by reducing the required time and/or energy. Furthermore, for aptamer based and other reversible sensors, the hydrophobic barrier must in some applications fully and quickly allow the analyte to leave the sensor and return to the sample solution.

With further reference to FIGS. 11A and 11B, although hydrophobic barriers 1160 may be comprised of solid or semi-solid materials (polymers, greases, etc.), in some embodiments, the hydrophobic barrier may be comprised of a fluid supported by a solid scaffold, e.g., a hydrophobic track-etch membrane with its pores impregnated with castor oil. Furthermore, the hydrophobic barrier as described may possess properties such as outlined for the example materials listed in Table 1.

TABLE 1

| | Melting | | | Solubility in | | | | | | | Double Bonds |
|---|---|---|---|---|---|---|---|---|---|---|---|
| | Point | | logP | Water | | Boiling Point | | Vapor Pressure | | Carbons | (in chain) |
| Oleic Acid | 61.3° | F. | 7.64 | 10 | μg/L | 547° | F. | 0.0000005 | mmHg | 18 | 1 |
| Linoleic Acid | 23° | F. | 6.8 | 1.59 | mg/L | 445° | F. | 0.00000087 | mmHg | 18 | 3 |
| Palmitoleic Acid | 32° | F. | 6.4 | Low | | 285° | F. | 0.000067 to 1.7 mmHg | | 16 | 1 |
| Arachidonic Acid | −50° | C. | 7 | Negligible | | High | | ~0 | mmHg | 20 | 4 |
| Decanol | 44° | F. | 4.57 | 30 | mg/L | 446° | F. | 0.00851 | mm Hg | 10 | 0 |
| Castor Oil | −10 to −12° C. | | 17.72 | <0.001 | mg/mL | 313° | C. | ~0 | mmHg | 57 | 3 |
| Tetradecane | 6° | C. | 8.19 | 0.00091 to | | 253° | C. | 0.0369 | mmHg | 14 | 0 |
| | | | | 0.0022 mg/L | | | | | | | |
| Mesitylene | 45° | C. | 3.4 | 0.0482 | mg/mL | 163-166° | C. | 2 | mmHg | 9 | 3 |
| Ricinoleic Acid | 5.5° | C. | 5.7 | 3460 | mg/L | 245° | C. | ~0 | mmHg | 18 | 1 |

| 10-Thiasteric Acid | | | | | | | 21 | 6 |
|---|---|---|---|---|---|---|---|---|
| Eicosenoic Acid | 23° | C. | 8.76 | 0.00096 to | ~0 | mmHg | 20 | 1 |
| | | | | 0.0019 mg/L | | | | |

| | | | | | | | | |
|---|---|---|---|---|---|---|---|---|
| Phytanic Acid | 8.3 | 0.002 to | ~7.5° | C. | ~0 | mmHg | 20 | 0 |
| | | 0.0068277 mg/L | | | | | | |

| Myristoleic Acid | | | 5.1 | 0.94 to | | ~0 | mmHg | 14 | 1 |
|---|---|---|---|---|---|---|---|---|---|
| | | | | 2.3128 mg/L | | | | | |
| Parinaric Acid | | | 5.9 | | | ~0 | mmHg | 18 | 4 |
| 2-Linoleoyl | | | 5.6 | 0.030 to | | ~0 | mmHg | 21 | 2 |
| Glycerol | | | | 0.56 mg/L | | | | | |
| Myristelaidic Acid | | | 5.1 | 0.002 | g/L | ~0 | mmHg | 14 | 1 |
| Anacardic Acid | 93° | C. | 9.5 | ~0.0005914 | mg/L | ~0 | mmHg | 22 | 3 |

With reference to Table 1, which lists log P values for water/octanol, although log P is typically used to characterize fluids, it is used here more broadly to interpret the effectiveness of a hydrophobic barrier, even if the hydrophobic barrier is not a liquid, is multilayered, multi-materialled, or some other deviation from a simple fluid. The disclosed invention may benefit from hydrophobic barrier with a log P that is at least one of >−1, >1, >3, or >5. Referenced herein as “analyte log P”, a log P value can also be measured with respect to analyte concentrations found in the sample solution 1180, sensor solution 1140, or hydrophobic barrier 1160. To maximize speed of transport for an analyte into and out of the device, the analyte log P will be between at least one of −1 and 1, −3 and 3, and −5 and 5.

Using log P to interpret the effectiveness of a hydrophobic barrier, consider an oil fluid having a water solubility of 50 mg/100 g (50 μg/g) as the hydrophobic barrier. If this oil fluid were embedded in a membrane that is 10% porous by volume and 10 μm thick, then the effective thickness of the oil fluid is ˜1 μm. Next, assume a 10 μm thick sample solution adjacent to the oil fluid that is flowed over the entire device (e.g., a continuous sweat biosensing device) so that new sample fluid is brought to the sensor every 10 minutes. Fresh sample fluid could then be brought to the sensor a total of 0.1*100/50E-3=200 times before the oil fluid is depleted (i.e., dissolved fully into the sample solution). The device would therefore last 33.3 hours before the oil fluid is depleted. The disclosed invention may therefore include a hydrophobic barrier at least partially comprised of a fluid with solubility limits in the sample solution that are at least one of <500 μg/g, <50 μg/g, <5 μg/g, <0.5 μg/g, resulting in hydrophobic barrier lifetimes of at least one of >3 hours, >30 hours, >300 hours, or >3000 hours.

With further reference to Table 1, and FIGS. 11A and 11B, hydrophobic barrier 1160 thickness will influence device operation. If the hydrophobic barrier is too thick, it can behave as a sink or storage location for the target analyte, and may lengthen the diffusive pathway the target analyte must traverse to reach the sensor 1120. Therefore, hydrophobic barrier thickness may be at least one of <1 mm, <100 μm, <10 μm, <1 μm, <0.1 μm.

With further reference to Table 1, and FIGS. 11A and 11B, hydrophobic barrier 1160 porosity will influence operation of the device. For example, a Teflon membrane or track etch membrane may be used as a solid scaffold to support an oil fluid, creating the hydrophobic barrier. If the porosity is low, target analyte diffusion and transport will be limited. Therefore, the hydrophobic barrier may have a porosity that is at least one of >0.1%, >1%, >10%, >30%.

With reference to Table 1, and FIGS. 11A and 11B, sensor solution 1140 thickness will influence proper device operation. The greater the thickness of the sensor solution, the longer the diffusive pathway to the sensor 1120, and the larger the volume that must be equilibrated with analyte concentration in the sample solution 1180. Sensor solution thickness therefore is at least one of <3 mm, <1 mm, <300 μm, <100 μm, <30 μm, or <10 μm.

With reference to Table 1, FIGS. 11A and 11B, the device will exhibit a concentration lag time that represents the time required for a concentration change in the sensor solution 1140 to reach 90% of the concentration in the sample solution 1180. Given cortisol as the target analyte, a sensor solution with a thickness of 100's of μm, and a hydrophobic barrier 1160 comprising a track-etch membrane of 10% porosity filled with castor oil, the concentration lag time can be on the order of 30 minutes. Optimizing the parameters above (log P, oil choice, thicknesses, etc.) results in concentration lag times that are at least one of <300 minutes, <100 min., <30 min., <10 min., <3 min., or <1 min.

With reference to Table 1, and FIGS. 11A and 11B, two or more oils may be blended in miscible form to obtain an oil fluid with optimal properties as outlined above. For example, additional properties may include a wider operating temperature range for an oil blend. Oils may also be immiscible, for example a 10 μm track etch membrane is filled with a first oil that is partially evaporated to form 2 μm thick plugs, then a second oil is added on one or both sides of the first oil plugs to form a hydrophobic barrier that is at least partially comprised of a plurality of immiscible oils. As configured, multiple immiscible oils may provide superior blocking of hydrophilic solute compared to a single oil (e.g., one oil may be better at blocking one type of hydrophilic solutes than the other). Furthermore, this may allow for more energy-favorable stepping of analyte transport (e.g., Oil 1: log P=2, Oil 2: log P=4, then Oil 3: log P=2, which is more favorable than directly bridging the energy gap created by a single oil with log P=4). Oil or fluids inside the hydrophobic membrane may also incorporate at least one solute that alters their log P values or some other property, and/or may include at least one surfactant which aids transport into or out of the oil fluid. For example, one or more phospholipids could affect the oil as a solute and/or as a surfactant.

With reference to Table 1, and FIGS. 11A and 11B, the disclosed invention may include a hydrophobic barrier 1160 that contains an oil with a low viscosity to allow an analyte to rapidly diffuse through the oil. Because of this relative ease of analyte diffusion, lower viscosities are desired. Therefore fluid for use in the hydrophobic barrier has a viscosity that is at least one of <1000 cP, <100 cP, or <10 cP. Example hydrophobic barrier oils include castor oil (viscosity of 650 centipoise (cP)) and dodecane (viscosity of 1.4 cP).

With further reference to Table 1, and FIGS. 11A and 11B, analytes that are ionized by factors such as pH in the sample solution 1180 or sensor solution 1140 will have greater difficulty in transporting through the hydrophobic barrier 1160. Potential of hydrogen can be controlled by buffering solutes. Therefore, the sample solution and/or sensor solution may contain at least one solute that maintains the analyte in an uncharged state. As an example, Tobramycin can be charged to a biological pH range (e.g., 6.8 to 7.2) and the sample solution buffered to a similar pH so that the uncharged Tobramycin can traverse the hydrophobic barrier for detection by the sensor 1120.

With further reference to FIGS. 11A and 11B, hydrophobic barrier 1160 could be a solid layer of a polymer, such as a 5 μm thick layer of polymethyldisiloxane (PDMS), which is permeable to certain target analytes, like cortisol. Silicone polymers reconfigure molecularly at a higher rate than other polymers, and therefore are able to transport hydrophilic analytes. However, silicone polymers may still function as a hydrophobic barrier as used herein, so long as the barrier adequately rejects interfering solutes.

With reference to FIG. 11B, the device 1100 is placed into contact with a sample solution 1180 such as blood, tears, sweat, interstitial fluid, urine, environmental (e.g., river) water, food product solution, or other types of sample solutions. The volume of sample solution could be small, e.g., from a single droplet to a ˜10 μm thick film, or large, e.g., a cup or more of fluid. Because water and other hydrophilic solvents can still diffuse, albeit slowly, through a hydrophobic barrier, the sensor solution 1140 could be contaminated by water or other solvents. One solution is to increase the atmospheric pressure of the sensor solution through applied pressure, osmosis, or other means. For example, the sensor 1120 could be an electrochemical aptamer based sensor for measuring cortisol in sweat, and the sensor solution may be a stabilizing solution of at least 1M MgCl with a fixed pH. If the hydrophobic barrier were adequately rigid or supported by a rigid material such as a stainless steel mesh, and the hydrophobic barrier is permeable to both cortisol and water, then even if sample solution has 10× lesser osmolarity than sensor solution, water would not be able to diffuse into sensor solution by osmosis because the volume of sensor solution is physically constrained by the rigid hydrophobic barrier. Similarly, the device could be constructed under a pressurized condition, e.g., 2× a standard atmosphere (atm), so that this built-in pressure reduces the amount of water able to enter or leave the sensor solution when the hydrophobic barrier interacts with the sample solution. Therefore, pressure or osmolality for the sensor solution could be at least one of 1.5×, 2×, or 10× greater than or less than pressure of the sample solution. Similarly, salinity of the sample solution could be at least one of 1.5×, 5×, 10×, or 100× different from salinity of the sensor solution. Furthermore, pH of the sample solution could be at least one of >0.5, >1, >2, or >5 pH units different from salinity of the sensor solution.

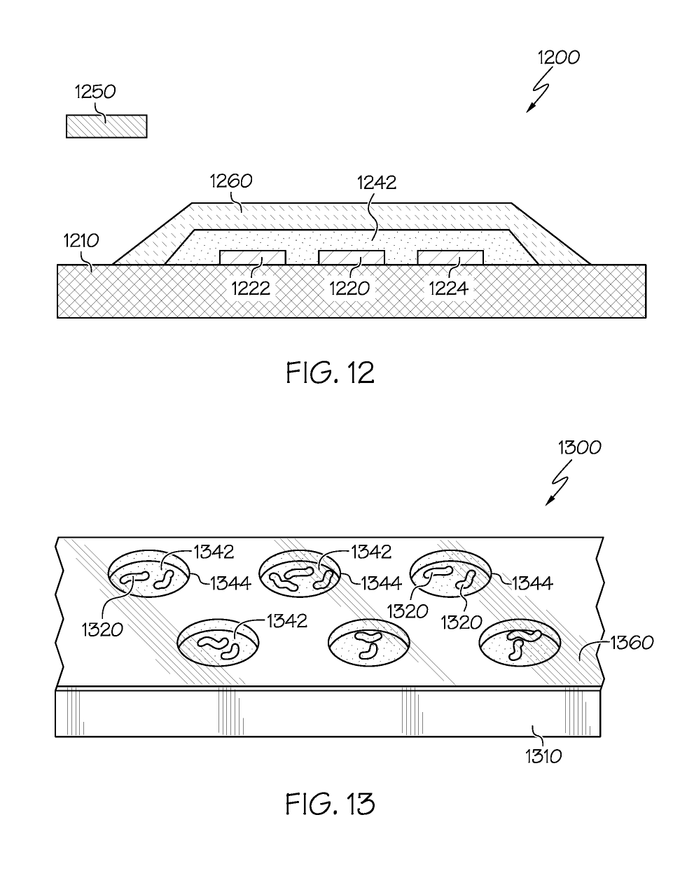

With reference to FIG. 12, where like numerals refer to like features previously described for FIG. 11A, the hydrophobic barrier 1260 could also be comprised of at least one membrane or other complex of lipid or phospholipid molecules similar to those found in cellular membranes. These molecules or membranes can be arranged in in monolayer, double layer, or a plurality of layers. For example, stacked droplets or micelles could comprise the hydrophobic barrier. The described lipid membranes and molecule complexes may be configured to comprise a very thin hydrophobic barrier, which promotes rapid diffusion and high diffusive flux of hydrophobic analytes.

With further reference to FIG. 11B, because of its electrical insulating properties, the hydrophobic barrier 1160 could potentially block DC electrical current and hinder sensor 1120 operation. Therefore, an electrically insulating hydrophobic barrier material may be configured to promote electrical conductivity, for example, by embedding PDMS with conductive nanoparticles, nanowires, meshes of carbon, metal, or PEDOT, or by embedding other electrically conductive materials. Alternately, if the hydrophobic barrier is too electrically resistive to promote direct charge transfer, charge could be capacitively coupled to the barrier using alternating voltages; that is, the hydrophobic barrier is at least in part an electrical capacitor with one or more electrodes 1150 outside the barrier and one or more sensors or electrodes inside the barrier.

With further reference to FIG. 12, the device 1200 may include a plurality of sensors or electrodes 1220, 1222, 1224, enclosed by the hydrophobic barrier 1260. Since readings by a reference electrode 1250 can fluctuate with changing sample solution properties, such as pH or salinity, it may be advantageous to seal a reference sensor and working and counter electrodes inside the hydrophobic barrier. This sensor configuration also eliminates the need for an electrically conductive hydrophobic barrier.

With further reference to FIG. 12, another issue that may arise with the use of a hydrophobic barrier 1260 is that a thin sensor solution 1240 can cause increased electrical resistance to develop between sensor electrodes, degrading sensor function. For example, if two or more sensors 1220, 1222, 1224 have shared electrodes, or electrodes that work together for electrochemical sensing (e.g., working and counter electrodes), then a thin sensor solution could raise electrical resistance between the electrodes and hamper sensor function. Therefore a plurality of sensor electrodes may be interdigitated, or may be otherwise configured to have a physical distance between their edges that is at least one of <100×, <10×, or <1× of the physical distance between the hydrophobic barrier and the electrodes.

With further reference to FIGS. 11A, 11B and 12, a hydrophobic barrier 1160, 1260 as described presents concrete advantages for sensor function in electrically noisy biofluids. For example, for electrochemical enzymatic sensors and aptamer sensors, biofluid samples of interest contain a large amount of untargeted solutes that are redox active and increase the background electrical current or noise. Fortunately, the majority of these untargeted solutes are charged or hydrophilic so that they are unable to diffuse through a hydrophobic barrier, thereby decreasing the background electrical current for a sensor in the sensor solution 1240 by at least 2×, 5×, 10×, 100×, or 1000× compared to background electrical noise in the sample solution 1280.

With further reference to FIGS. 11A and 11B, if the hydrophobic barrier 1160 is oil-based or another material that could foul or damage the sensor 1120, some embodiments of the disclosed device may further comprise a hydrophilic coating 1140, such as sucrose or a collagen hydrogel, to protect the sensor from fouling by the hydrophobic barrier during use or fabrication. In other embodiments, the sensor may also include a spacer, e.g., a plurality of patterned pillars of SU-8 photo-definable epoxy, that separate the hydrophobic barrier from the sensor surface to prevent contact between the sensor and hydrophobic materials. The disclosed invention may therefore include one or more hydrophilic coatings or spacers between the hydrophobic barrier and the sensor.

With reference to FIG. 13, where like numerals refer to like features found in FIGS. 11A, 11B and 12, a device 1300 has solvent 1342 and sensor or sensing material 1320 that is fully enclosed by a hydrophobic barrier 1360. For example, the sensing material is an optical aptamer probe for testosterone, and the solvent is an aqueous solution. When the device is exposed to a sample, testosterone from the sample diffuses through the hydrophobic barrier to the solvent and sensing material. Binding of testosterone to the sensing material causes a change in the optical transmission wavelength (colorimetric) or optical fluorescence (fluorometric) of the sensor. Further, these embodiments also encapsulate the sensor and solvent in a hydrophobic barrier that reduces or prevents diffusion of ions or other charged or hydrophilic species. Therefore, such embodiments benefit from the same advantages described previously for membrane-enhanced sensors, namely improved responsiveness to target analyte concentrations, with less sensitivity to pH or salinity fluctuations, or other confounding factors.

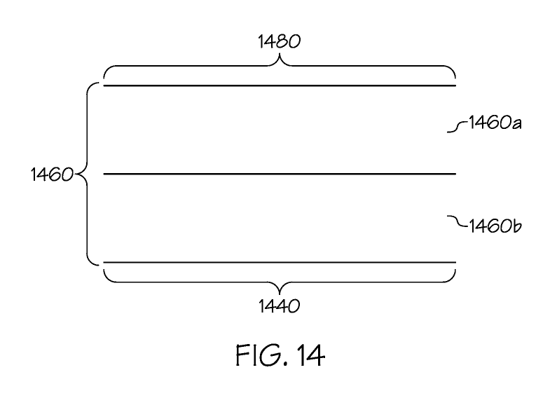

With reference to FIG. 14, where like numerals refer to like features found in FIGS. 11A, 11B, 12 and 13, a hydrophobic barrier 1460 could be comprised of a first layer 1460a, such as a silicone polymer, and a second layer 1460b, such as a track etch membrane with a volatile oil. The polymer layer 1460a prevents evaporation of the volatile oil layer 1460b, while the volatile oil layer functions to block hydrophilic solutes from interacting with the sensor. Therefore, the hydrophobic barrier may include a plurality of layers, and among the plurality of layers are alternating layers of one or more solid materials and one or more fluid materials.

This has been a description of the disclosed invention along with a preferred method of practicing the invention, however the invention itself should only be defined by the appended claims.

What is claimed is:

1. A device, comprising:
one or more sensors configured to measure a characteristic of an analyte in a biofluid, wherein the analyte comprises a bound fraction that is chemically bound to a binding solute, and further comprises an unbound fraction that is not chemically bound to the binding solute, and wherein at least one sensor is configured to measure a characteristic of the bound fraction; and
one or more analyte releasers configured to cause at least a portion of the bound fraction to release from the binding solute.

2. The device of claim 1, wherein in the characteristic is a concentration.

3. The device of claim 1, further comprising a biofluid collector.

4. The device of claim 1, further comprising one or more sample conduits.

5. The device of claim 1, wherein the one or more analyte releasers causes a percentage of the bound analyte to release from the binding solute, wherein the percentage is one of the following: greater than (>) 30%, >60%, >90%, or >95%.

6. The device of claim 1, wherein said analyte releaser further comprises one of the following: an electric heater; a solvent; an energy source; an electrode; or a solute introducer.

7. The device of claim 6, wherein the analyte releaser is a solute introducer that adds a solute to the biofluid that alters one of the following characteristics of the biofluid: a potential of hydrogen (pH) value; or a salinity value.

8. The device of claim 6, wherein the analyte releaser is a solute introducer that adds a solute to the biofluid that competes with the analyte for binding with the binding solute.

9. The device of claim 6, wherein the analyte releaser is an electrode that alters a pH value of the biofluid.

10. The device of claim 1, wherein the analyte releaser further comprises a volume, and wherein the volume is one of the following: less than (<) 10 mL, <3 mL, <1 mL, <0.3 mL, or <0.1 mL.

11. The device of claim 1, wherein the analyte releaser is configured to operate for one of the following periods: greater than (>) 1 hour, >12 hours, >24 hours, or >3 days.

12. The device of claim 1, wherein the one or more sensors comprises one or more of the following: an electrochemical aptamer-based sensor; an electrochemical enzyme-based sensor; a continuous sensor; or a reversible sensor.

13. The device of claim 1, further comprising one or more pumps configured to draw the biofluid into or through the device.

14. The device of claim 1, wherein the one or more sensors and the analyte releaser are co-located.

15. The device of claim 1, further comprising a plurality of fluid pathways.

16. The device of claim 15, wherein one of the plurality of fluid pathways removes an excess amount of binding solute from the device.

17. The device of claim 1, further comprising one or more sensor protectors configured to protect at least one of the sensors from effects caused by the one or more analyte releasers.

18. The device of claim 17, wherein the sensor protector further comprises includes one or more filters configured to remove a portion of the binding solute from the biofluid, and one or more reverters configured to reverse the effects on the biofluid caused by the one or more analyte releasers.

19. The device of claim 17, wherein the one or more sensor protectors further comprise one or more components configured to alter a characteristic of the biofluid.

20. The device of claim 17, wherein the sensor protector further comprises one or more hydrophobic barriers.

21. The device of claim 1 wherein the one or more sensors includes a first sensor configured to measure a first characteristic of the bound fraction of the analyte, the unbound fraction of the analyte, or a total fraction of the analyte, and a second sensor configured to measure a second characteristic of the bound fraction of the analyte, the unbound fraction of the analyte, or the total fraction of the analyte.

22. The device of claim 2, wherein the one or more sensors is configured to measure two or more of: a concentration of the bound fraction of the analyte, a concentration of the unbound fraction of the analyte, and a concentration of a total fraction of the analyte.

23. The device of claim 1 wherein a characteristic of a plurality of analytes are measured.

24. The device of claim 1, further comprising a needle, and wherein the biofluid is one of the following: an interstitial fluid sample, or a blood sample.

25. The device of claim 1, wherein the device is configured as one of the following: a capsule, an implant; a reusable device; a disposable device.
